(19) United States
(12) Patent Application Publication
(10) Pub. No.: US 2009/0060972 A1
(43) Pub. Date: Mar. 5, 2009

(54) DEVICE AND METHOD FOR CELL GRAFTING

(75) Inventor: Alon Shalev, Raanana (IL)

Correspondence Address: BROWDY AND NEIMARK, P.L.L.C.;624 NINTH STREET, NW, SUITE 300, WASHINGTON, DC, 20001-5303, US

(73) Assignee: NICAST LTD., Lod (IL)

(21) Appl. No.: 12/094,523

(22) PCT Filed: Nov. 21, 2006
(86) PCT No.: PCT/IL2006/001336

§ 371 Date: Nov. 3, 2008

Related U.S. Application Data
(60) Provisional application No. 60/738,044, filed on Nov. 21, 2005.

(51) Int. Cl. A61F 2/00; A61M 31/00

(52) U.S. Cl. 424/423; 604/57

(57) ABSTRACT

A device for cell grafting is disclosed. The device comprises a compartment enclosed by a semipermeable envelope made at least in part of nonwoven polymer fibers, and at least one inlet port formed in the envelope. The inlet port(s) are accessible from the exterior of the envelope and configured for establishing fluid communication with the compartment.

### FIELD AND BACKGROUND OF THE INVENTION

The present invention relates to medical devices and, more particularly, but not exclusively, to a device and method for cell grafting.

Local delivery of biological and biochemical substances is used in treating damaged, traumatized, abnormal functioning, diseased and/or dysfunction tissues. It is recognized that local delivery of biological and biochemical substances can restore or compensate for the impairment or loss of organ or tissue function. In many disease or deficiency states, the affected organ or tissue is one which normally functions in a manner responsive to fluctuations in the levels of specific metabolites, thereby maintaining homeostasis. For example, the parathyroid gland normally modulates production of parathyroid hormone in response to fluctuations in serum calcium. Similarly, beta-cells in the pancreatic islets of Langerhans normally modulate production of insulin in response to fluctuations in serum glucose.

However, traditional therapeutic approaches to the treatment of such diseases cannot compensate for the responsiveness of the normal tissue to these fluctuations. For example, an accepted treatment for diabetes includes daily injections of insulin. This regimen cannot compensate for the rapid, transient fluctuations in serum glucose levels produced by, for example, strenuous exercise. Failure to provide such compensation may lead to complications of the disease state; this is particularly true in diabetes.

Similarly, presently available techniques for intraspinal administration of medications for modulating pain sensitivity in the spinal cord, have been associated with various complications and undesired neurological syndromes. In the area of brain therapy, attempts have been made to devise miniature osmotic pumps which locally provide a constitutive supply of drugs or other factors to the brain. However, limited solubility and stability of certain drugs, as well as reservoir limitations, have restricted the usefulness of this technology. Another technique employs microcapsules or macroencapsulation which encapsulate a microscopic droplet of a cell solution, for therapeutic implantation purposes. Yet, there are shortcomings in these approaches because the viability of the encapsulated cells as in vivo implants often fails. Even when the cells remain viable, they sometimes secrete their products at lower than therapeutically useful levels.

Strategies for local delivery biological and biochemical substances are therefore being developed in response to a range of clinical needs. One such local delivery technique includes the transplantation of viable cells near the target tissue. The viable cells produce and secrets the desired biological or biochemical substances which restore or compensate for the impairment of the tissue function. It is recognized, however, that although such transplantation can provide dramatic benefits, it is limited in its application by the relatively small number of organs suitable and available for grafting. Generally, the subject is immunosuppressed prior to the transplantation so as to avert immunological rejection of the transplant, which results in loss of transplant function and eventual necrosis of the transplanted tissue or cells. In many cases, the transplanted cells must remain functional for a long period of time, even for the remainder of the patient's lifetime. It is both undesirable and expensive to maintain a patient in an immunosuppressed state for a substantial period of time.

Known in the art are transplantation procedures in which cells or tissues are is implanted within a physical barrier which allows diffusion of nutrients, waste materials, and secreted substances, but block the cellular and molecular effectors of immunological rejection (see, e.g., U.S. Pat. Nos. 4,892,538, 5,106,627, 5,156,844, 5,182,111, 5,798,113, 5,800,828, 5,800,829, 5,834,001, 5,869,077, 5,871,767, 5,874,099, 6,083,523, 6,322,804, 6,960,351).

A promising manufacturing technique for implantable devices is electrospinning. Electrospinning is a method for the manufacture of ultra-thin synthetic fibers which reduces the number of technological operations required in the manufacturing process and improves the product being manufactured in more than one way.

The process of electrospinning creates a fine stream or jet of liquid that upon proper evaporation of a solvent or liquid to solid transition state yields a nonwoven structure. The fine stream of liquid is produced by pulling a small amount of polymer solution through space by using electrical forces. More particularly, the electrospinning process involves the subjection of a liquefied substance, such as polymer, into an electric field, whereby the liquid is caused to produce fibers that are drawn by electric forces to an electrode, and are, in addition, subjected to a hardening procedure. In the case of liquid which is normally solid at room temperature, the hardening procedure may be mere cooling; however other procedures such as chemical hardening (polymerization) or evaporation of solvent may also be employed. The produced fibers are collected on a suitably located precipitation device and subsequently stripped from it. The sedimentation device is typically shaped in accordance with the desired geometry of the final product, which may be for example tubular, flat or even an arbitrarily shaped product.

Electrospinning has known to be employed for manufacturing or coating of implantable medical devices, particularly vascular prostheses and stents. The technique permits to obtain a wide range of fiber thickness (from tens of nanometers to tens of micrometers), achieves exceptional homogeneity, smoothness and desired porosity distribution along the coating thickness.

Despite the evolution of numerous artificial or synthetic grafts that have been developed, the capability of known techniques to adequately substitute for nature's highly complex structures and to overcome the body's defense system, is far from being satisfactory. Despite major advancements in this field, modern techniques remain associated with complications including inflammation, degradation and rejection. Furthermore, presently known techniques have not been satisfactory for providing long-term transplant function.

There is thus a widely recognized need for, and it would be highly advantageous to have a device and method for cell grafting devoid of above limitations.

### SUMMARY OF THE INVENTION

According to one aspect of the present invention there is provided a device for cell grafting. The device comprises a compartment enclosed by a semipermeable envelope made at least in part of nonwoven polymer fibers, and at least one inlet port formed in the envelope. The inlet port(s) are accessible from the exterior of the envelope and configured for establishing fluid communication with the compartment.

According to further features in preferred embodiments of the invention described below, the device comprises a sufficient amount of viable cells.

According to yet another aspect of the present invention there is provided a method of delivering a biologically active substance to a mammal. The method comprises delivering into the compartment of the device a sufficient amount of viable cells which are capable of secreting the biologically active substance, and introducing the device to a location requiring the biologically active substance.

According to further features in preferred embodiments of the invention described below, the delivery of the viable cells is performed prior to the step of introducing the device to the location.

According to still further features in the described preferred embodiments the sufficient amount is at least 10,000 cells.

According to still further features in the described preferred embodiments the introducing the viable cells is performed while the device is at the location.

According to still further features in the described preferred embodiments the method further comprises harvesting the viable cells from a mammalian donor.

According to still further features in the described preferred embodiments the location is in the body of a mammal.

According to still further features in the described preferred embodiments the device is introduced by percutaneously injection.

According to still further features in the described preferred embodiments the method further comprises imaging at least a part of the body during the step of introducing the device to the location.

According to still further features in the described preferred embodiments the method further comprises monitoring the device in vivo.

According to still further features in the described preferred embodiments the semipermeable envelope comprises a plurality of layers.

According to still further features in the described preferred embodiments the inlet port(s) is generally closed and has a self sealing capability following piercing.

According to still further features in the described preferred embodiments inlet port(s) comprises non-woven swellable polymer fibers.

According to still further features in the described preferred embodiments the device further comprises one or more outlet ports formed in the envelope.

According to still further features in the described preferred embodiments the outlet port(s) is hydrophilic.

According to still further features in the described preferred embodiments the outlet port(s) is designed and configured for maintaining fluid homeostasis between the compartment and the environment.

According to still further features in the described preferred embodiments the semipermeable envelope is characterized by a molecular weight cutoff from about 75 kDa to about 500 kDa.

According to still further features in the described preferred embodiments the semipermeable envelope comprises at least one pharmaceutical agent incorporated therein.

According to still further features in the described preferred embodiments the pharmaceutical agent(s) comprises antibodies immobilized upon the semipermeable envelope.

According to still further features in the described preferred embodiments the pharmaceutical agent(s) comprises a diagnostic agent, e.g., an imaging agent.

According to still further features in the described preferred embodiments the pharmaceutical agent(s) comprises a pro-angiogenic agent.

According to still further features in the described preferred embodiments the pharmaceutical agent(s) comprises a vascular endothelial growth factor.

According to still further features in the described preferred embodiments the pharmaceutical agent(s) comprises an antibiotic agent.

According to still further features in the described preferred embodiments the pharmaceutical agent(s) comprises an immuno-suppressing agent.

According to still further features in the described preferred embodiments the device further comprises at least one regulatory compound. According to still further features in the described preferred embodiments the regulatory compound(s) comprises a potentiating compound selected to increase and/or mediate the ability of the growth factor to regulate or mediate at least one of: cell proliferation, cell differentiation, tissue regeneration and cell attraction. According to still further features in the described preferred embodiments the regulatory compound(s) comprises an inhibiting compound selected to inhibit agents interfering with ability of the growth factor to regulate or mediate at least one of: cell proliferation, cell differentiation, tissue regeneration and cell attraction.

According to still further features in the described preferred embodiments the cells are selected from the group consisting of insulin producing cells, adrenal chromaffin cells, antibody-secreting cells, fibroblasts, astrocytes and beta cell lines. According to still further features in the described preferred embodiments the insulin producing cells are in the form of Islets of Langerhans.

According to still further features in the described preferred embodiments the cells are capable of secreting a biologically active substance.

According to still further features in the described preferred embodiments the cells are capable of providing a biological function to a subject.

According to still further features in the described preferred embodiments the biologically active substance comprises an analgesic or a pain-reducing substance. According to still further features in the described preferred embodiments the pain-reducing substance is selected from the group consisting of enkephalins peptide, catecholamine peptide, opioid peptide and any mixture thereof.

According to still further features in the described preferred embodiments the biologically active substance comprises at least one of: a growth factor and a trophic factor. According to still further features in the described preferred embodiments the growth factor is selected from the group consisting of a nerve growth factor, a basic fibroblast growth factor, a platelet derived growth factor and an epidermal growth factor. According to still further features in the described preferred embodiments the trophic factor comprises a neurotrophic factor. According to still further features in the described preferred embodiments the neurotrophic factor is selected from the group consisting of a ciliary neurotrophic factor, a brain-derived neurotrophic factor and a glial-derived neurotrophic factor. According to still further features in the described preferred embodiments the neurotrophic factor comprises neurotrophin-3.

According to still further features in the described preferred embodiments the biologically active substance comprises a hormone.

According to still further features in the described preferred embodiments the biologically active substance comprises cytokine.

According to still further features in the described preferred embodiments the biologically active substance comprises lymphokine.

According to still further features in the described preferred embodiments the biologically active substance comprises a neurotransmitter. According to still further features in the described preferred embodiments the neurotransmitter is selected from the group consisting of dopamine, L-dopa, gamma aminobutyric acid, serotonin, acetylcholine, noradrenaline, epinephrine, glutamic acid.

According to still further features in the described preferred embodiments the biologically active substance comprises a neuropeptide. According to still further features in the described preferred embodiments the neuropeptide comprises at least one neurotensin and Substance P.

According to still further features in the described preferred embodiments the biologically active substance comprises at least one substance selected from the group consisting of: parathyroid hormone, interleukin, erythropoietin, albumin, transferrin and Factor VIII.

The present invention successfully addresses the shortcomings of the presently known configurations by providing a device for cell grafting and a method for a method of delivering a biologically active substance to a mammal.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are schematic fragment illustrations of a device for cell grafting, according to various exemplary embodiments of the present invention;

FIG. 2 is a schematic fragment illustration showing a layer of an envelope of the device, in a preferred embodiment in which the layer is formed of a non-woven web of polymer fibers having therein embedded particles constituting a substance;

FIG. 3 is a schematic fragment illustration showing a layer of an envelope of the device, in a preferred embodiment in which the layer is comprises compact objects distributed between the polymer fibers;

FIG. 4 is a flowchart diagram of a method for cell grafting, according to various exemplary embodiments of the present invention.

### DESCRIPTION OF THE PREFERRED EMBODIMENTS

The present invention is of a device and method which can be used for cell grafting. Specifically, the present invention can be used for delivering a biologically active substance and/or providing metabolic functions to a target tissue.

The principles and operation of a device and method according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As used herein, “cell grafting” describes applications which relate to biological substitutes to restore, maintain or improve tissue functions. More specifically, “cell grafting” encompasses any process wherein viable cells are delivered to a specific location and optionally being cultured at the specific location to restore, maintain or improve tissue functions.

The present embodiments provide a the device which can function as a vehicle for delivering one or more biologically active substances to a location requiring such substances. The device can serve for of altering cell growth, bone growth, nerve stimulation, biological response or a combination thereof. It can also serve for providing a metabolic capability or function, such as the removal of specific solutes from the bloodstream.

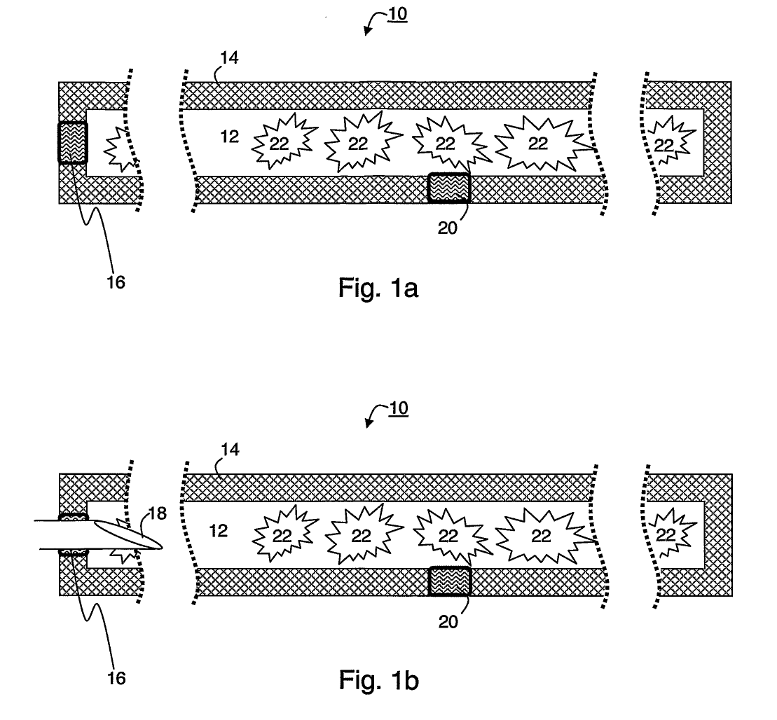

Referring now to the drawings, FIGS. 1a-b illustrate a device 10 for cell grafting, according to various exemplary embodiments of the present invention. Device 10 comprises a compartment 12 enclosed by a semipermeable envelope 14, and one or more inlet ports 16 formed in envelope 14.

The term “semipermeable envelope” as used herein refers to a structure such as a membrane allowing the diffusion therethrough of solutes having a molecular weight which is less than or equals a predetermined molecular weight cutoff, and prevents the diffusion therethrough of solutes having other molecular weights. Preferred molecular weight cutoffs according to various exemplary embodiments of the invention are from about 75,000 Daltons (75 kDa) to about 500,000 Daltons (500 kDa). Such values of molecular weight cutoffs allows the diffusion of various biologically active substances (e.g., growth factors, trophic factors, hormones, neurotransmitters, sufficiently small peptides, etc.), and prevent diffusion of various cells (e.g., insulin producing cells, adrenal chromaffin cells, antibody-secreting cells, fibroblasts, astrocytes, beta cells, etc.).

The advantage of using a semipermeable envelope is that the anterior of compartment 12 is protected by envelope 14 from immunological attack while device 10 is implanted at an implantation location in vivo. Although some low molecular weight mediators of the immune responses (e.g., cytokines which are known to have a molecular weight of less than 70 kDa) may be permeable to the envelope 14, in most cases local or circulating levels of these substances are not high enough to have detrimental effects. The anterior of compartment 12 is protected from attack by the recipient's immune system and from potentially deleterious inflammatory responses from the tissues which surround the device at the implantation location. Thus, compartment 12 can be occupied by various substances 22 (e.g., viable cells) which otherwise (i.e., in the absence of envelope 14) would be attacked by immune system. For example, compartment 12 can be occupied or partially occupied by viable tissue which is allogeneic and even xenogeneic to the recipient, without being rejected by the recipient's immune system. In this manner, needed substances or metabolic functions can be delivered to the recipient even for extended periods of time, and without the need to treat the recipient with dangerous immunosuppressive drugs.

Envelope 14 and/or port 16 are preferably made, at least in part, of biocompatible nonwoven polymer fibers.

The term “polymer”, as used herein includes, but is not limited to, homopolymer, copolymer, e.g., block, graft, random and alternating copolymer, terpolymer, etc., and blends and/or modifications thereof. Furthermore, unless otherwise specifically limited, the term “polymer” includes all possible geometrical configurations, including, without limitation, isotactic, syndiotactic and atactic symmetries.

The polymer fibers of envelope 14 can be made of a biostable or a biodegradable polymer as desired. Biostable polymer suitable for the present embodiments can comprise one or more of the following polymers: polycarbonate based aliphatic polyurethanes, siloxane based aromatic polyurethanes, polydimethylsiloxane and other silicone rubbers, polyester, polyolefins, polymethyl-methacrylate, vinyl halide polymer and copolymers, polyvinyl aromatics, polyvinylidene fluoride, polyvinyl esters, polyamides, polyimides and polyethers.

Biodegradable polymer suitable for the present embodiments can comprise one or more of the following polymers: poly(L-lactic acid), poly(D-lactic acid), poly (lactide-co-glycolide), polycaprolactone, polyphosphate ester, poly (hydroxybutyrate), poly (glycolic acid), poly (DL-lactic acid), poly (amino acid), cyanocrylate, copolymers and biomolecules such as, but not limited to, collagen, DNA, silk, chitozan and cellulose.

The use of biodegradable polymer is particularly useful when device 10 carries one or more pharmaceutical agents as further detailed hereinunder. The use of biodegradable polymer is also advantageous for controlling the interaction between device 10 and the body of an individual (a human or an animal subject) in whom device 10 is implanted. For example, envelope 14 can be made, at last in part, of hydrophilic water-degradable polymer fibers which start to degrade a predetermined period after implantation.

Port 16 is preferably accessible from the exterior of envelope 14 and is configured for establishing fluid communication with compartment 12. According to a preferred embodiment of the present invention port 16 is generally closed and has a self sealing capability following piercing. In use, a tip 18 of an injection device, such as a syringe needle or the like, temporarily penetrates through port 16 to allow fluid exchange between the injection device and compartment 12. Upon the withdrawal of tip 18, port 16 is self-sealed, for example, via swelling of the fibers forming port 16 and/or via reordering of the fiber at the penetration cite.

When the self-sealing is via swelling, port 16 preferably comprises non-woven water-swellable polymer fibers.

The polymer fibers of port 16 are preferably made of a water-swellable polymer.

The term “swellable” as used herein describes a functionality of a material to expand or increase in physical size (length, area and/or volume) in the presence of a swelling agent. The term “water-swellable polymer” refers to a swellable polymer for which the swelling agent is aqueous medium, such as, but not limited to, water or biological fluids. A “water-swellable polymer” preferably imbibes water with a concomitant increase in volume when exposed to the aqueous medium. Water swellable polymer generally retains its original identity or physical structure, but in a highly expanded state, during the absorption or adsorption of the water.

In various exemplary embodiments of the invention the water-swellable polymer is capable, under the most favorable conditions, of absorbing or adsorbing at least about 3 times its weight and, more desirably, at least about 6 times its weight in the presence of the swelling agent.

As used herein the term “about” refers to ±10%.

The water-swellable polymer of the present embodiments can be natural, synthetic or modified natural polymer. In addition, the water-swellable polymer of the present embodiments can be an inorganic polymer, or an organic polymer.

Representative examples of a water-swellable polymers suitable for the present embodiments, include, without limitation, modified polyurethanes such as those marketed under the trade names, Hydrothane™, Hydromed™ and Hydroslip™, polyacrylamide, polyvinyl alcohol, poly (hydroxyethyl methacrylate), poly (hydroxypropyl methacrylate), polyacrylate-polyalcohol and the like.

Water-swellable polymer fibers of the present embodiments can also comprise other materials, such as, but not limited to, poly (isobutylene-co-maleic acid) sodium salt, gelatin and collagen.

When the self-sealing is via reordering of the fibers, port 16 comprises at least one layer of nonwoven fibers which is characterized by sufficiently high elasticity (preferably more than 50%), sufficiently large number of inter-fiber voids (preferably 10,000 voids per cubic mm) and sufficiently small number of bonds between the fibers (preferably 1,000 voids per cubic mm).

In various exemplary embodiments of the invention device 10 further comprises one or more outlet ports 20 formed in envelope 14. Outlet port 20 is preferably designed and configured for maintaining fluid homeostasis between compartment 12 and the environment. According to a preferred embodiment of the present invention outlet port 20 is hydrophilic. Outlet port 20 can be made of nonwoven polymer fibers, preferably biostable nonwoven polymer fibers. Representative examples of polymers suitable for outlet port 20 include, without limitation, hydrophilic thermoplastic polyurethanes (Hydrothane, Elasthane, Elast-Eon), polysulfone, polycarbonate, polymethyl-methacrylate, polyvinyl esters and polyethers. In various exemplary embodiments of the invention port 20 is more rigid compared to envelope 14.

The non-woven polymer fibers can be formed by any fiber-forming process known in the art. In various exemplary embodiments of the invention a spinning technique is employed. The preferred spinning technique is the electrospinning technique, in which a fine stream or jet of liquid is produced by pulling a small amount of charged liquefied polymer through space using electrical forces. The produced fibers are hardened and collected on a suitably located precipitation device to form the nonwoven article of electrospun fibers. Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference.

It is to be understood that although the according to the presently preferred embodiment of the invention is described with a particular emphasis to the electrospinning technique, it is not intended to limit the scope of the invention to the electrospinning technique. Representative examples of other spinning techniques; suitable for the present embodiments include, without limitation, a wet spinning technique, a dry spinning technique, a gel spinning technique, a dispersion spinning technique, a reaction spinning technique or a tack spinning technique. Such and other spinning techniques are known in the art and disclosed, e.g., in U.S. Pat. Nos., 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

The typical thickness of the polymer fibers of envelope 14, is without limitation, from about 50 nm to about 2000 nm, more preferable from about 100 nm to about 500 nm. The overall thickness of envelope 14 is preferably from about 0.1 mm to about 1 mm, more preferably from about 0.5 mm to about 1 mm.

The typical thickness of the polymer fibers of inlet port 16, is without limitation, from about 100 nm to about 1,000 nm, more preferable from about 300 nm to about 400 nm. The typical thickness of the polymer fibers of outlet port 20, is without limitation, from about 100 nm to about 1,000 nm, more preferable from about 400 nm to about 600 nm. According to a preferred embodiment of the present invention the overall thickness of inlet port 16 and outlet port 20 is independently from about 20 μm to about 200 μm, more preferably from about 50 μm to about 100 μm.

Device 10 preferably, but not obligatorily, has a generally elongated shape. The preferred dimensions of device 10 are from about 1 cm to about 10 cm in length, and from about 100 μm² to about 1 mm² in cross sectional area. The advantage of using an elongated device is to facilitate the implantation of device 10 in the body of a mammalian host via percutaneous injection. It is to be understood that although an elongated shape of the above dimensions is the preferred shape of device 10 it is not intended to limit the scope of the present invention to any specific shape of device 10. Thus, there are many alternatives for the shape and/or dimensions of device 10, including, without limitation, a disc, an oval, a sphere, an ellipsoid a cuboid and the like.

In various exemplary embodiments of the invention device 10 comprises a sufficient amount (say, about 10,000 or more) of viable cells 22 which at least partially occupy compartment 12.

Unless otherwise specified, the term “cells” as used herein refers to cells in any form, including, but not limited to, cells retained in tissue, cell clusters and individually isolated cells.

Cells 22 are optionally and preferably capable of exerting a biologically useful effect upon the body of an individual (a human or an animal subject) in whom device 10 is implanted. According to a preferred embodiment of the present invention cells 22 secrete or release a biologically active substance.

The biologically active substance can be any active factor such as a neurotransmitter, neuromodulator, catecholamine, growth factor, cofactor, trophic factor and hormone. The biologically active substance can also be an analog, an agonist, a derivative or a fragment of an active factor having the biological activity of the active factor. The biologically active substance can further be an inhibitor of a normal biological factor. This embodiment is particularly useful in instances where a disease is caused by an excess of such biological factor (e.g., as in Huntington's Disease).

Cells 22 can include cells which naturally produce and secrete the biologically active substance, and/or cells which are genetically engineered to produce the biologically active substance.

It is expected that during the life of this patent many relevant biologically active materials will be developed and the scope of the term biologically active material is intended to include all such new technologies a priori.

Alternatively or additionally, cells 22 can provide a metabolic capability or function, e.g., removing of specific solutes from the bloodstream of the individual.

Compartment 12 and/or envelope 14 are preferably constructed to provide a suitable local environment for the continued viability and function of cells 22. Thus, according to a preferred embodiment of the present invention compartment 12 and/or envelope 14 comprise a culture medium, optionally containing a liquid source of additional factors to sustain cell viability and function. Such culture medium can include, for example, salts, sugars, amino acids and minerals in the appropriate concentrations and with various additives and those of skills in the art are capable of determining a suitable culture medium to specific cell types. For example, compartment 12 and/or envelope 14 can comprise natural or synthetic nutrient sources, extracellular matrix components, growth factors or growth regulatory substances, a population of feeder, accessory cells or O₂ carriers.

Cells 22 can be of any type suitable for exerting the biologically useful effect. Representative examples include, without limitation, fully-differentiated cells, anchorage-dependent cells, primary tissues, incompletely-differentiated fetal tissues, neonatal tissues, anchorage-independent transformed cells cell lines and the like. More specifically, but not exclusively, cells 22 can comprise insulin producing cells (e.g., Islets of Langerhans), adrenal chromaffin cells, antibody-secreting cells, fibroblasts (particularly fibroblasts which have been genetically engineered to produce recombinant nerve growth factor), astrocytes and/or beta cell lines.

Biologically active substances which can be delivered using device 10 include a wide variety of factors normally secreted by various organs or tissues. For example, insulin can be delivered to a diabetic patient, dopamine to a patient suffering from Parkinson's disease, or Factor VIII to a Type A hemophiliac.

Another family of biologically active substances suited to delivery by device 10 comprises biological response modifiers, including lymphokines and cytokines. Antibodies from antibody secreting cells may also be delivered. Specific antibodies which may be useful include those towards tumor specific antigens. The release of antibodies may also be useful in decreasing circulating levels of compounds such as hormones or growth factors. These products are useful in the treatment of a wide variety of diseases, inflammatory conditions or disorders, and cancers.

Cells 22 are preferably chosen for their secretion of hormones, cytokines, growth factors, trophic factors, angiogensis factors, antibodies, blood coagulation factors, lymphokines, enzymes, and other therapeutic agents or agonists, precursors, active analogs, or active fragments thereof. These include, without limitation, insulin, parathyroid hormone, interleukin 3, albumin, transferrin, factor VIII, erythropoietin, nerve growth factor, glial derived neurotrophic factor, platelet-derived growth factor, epidermal growth factor, brain-derived neurotrophic factor, neurotrophin-3, an array of fibroblast growth factors and ciliary neurotrophic factor. Also contemplated are peptides such as, but not limited to, endorphins, dynorphin and any mixture thereof, and neuropeptides such as, but not limited to, neurotensin and Substance P.

Trophic factors, for example, are particularly useful when device 10 is implanted to a proper brain region. Suitably, in this embodiment cells 22 are adrenal chromaffin cells which are known to secrete at least one factor, basic fibroblast growth factor. Other as yet unidentified trophic factors may also be secreted by chromaffin cells.

Cells 22 can also secrete or release various of neurotransmitters, include, without limitation, dopamine, L-dopa, gamma aminobutyric acid, serotonin, acetylcholine, noradrenaline, epinephrine, glutamic acid.

Neurotransmitters are small molecules (less than 1 kDa molecular weight) which act as chemical means of communication between neurons. They are synthesized by the presynaptic neuron and released into the synaptic space where they are then taken up by postsynaptic neurons. Neurotransmitter deficits have been implicated in various neurological diseases. Lack of neurotransmitter-mediated synaptic contact causes neuropathological symptoms, and can also lead to the ultimate destruction of the neurons involved. It is recognized that localized delivery of the relevant neurotransmitter to the target tissue may reverse the symptoms without the need for specific synaptic contact. The advantage of the presently preferred embodiment of the invention is that it facilitates delivery of the required neurotransmitter to a localized target region which is deficient in that neurotransmitter, without affecting other neurological structures.

The device of the present embodiments is also useful for treating individuals suffering from acute and/or chronic pain, by delivery of an analgesic or pain reducing substance to the individual. Such pain reducing substances include enkephalin peptide, catecholamine peptide, and other opioid peptides. Such compounds may be secreted by, e.g., adrenal chromaff in cells.

In various exemplary embodiments of the invention envelope 14 comprises at least one pharmaceutical agent incorporated therein.

As used herein “pharmaceutical agent” refers to a therapeutic agent (e.g., a medicament) or a diagnostic agent (e.g., an imaging agent).

The pharmaceutical agent can comprise, for example, an antibody immobilized upon envelope 14, a pro-angiogenic agent (e.g., a vascular endothelial growth factor), an antibiotic agent, an immuno-suppressing agent (e.g., rapamycin, a rapamycin analogue, or a rapamycin derivative), and the like.

Envelope 14 can further comprise one or more regulatory compounds, which preferably comprise a potentiating compound selected to increase and/or mediate the ability of the growth factor to regulate or mediate cell proliferation, cell differentiation, tissue regeneration, cell attraction and/or wound repair. Alternatively or additionally, the regulatory compound(s) comprise an inhibiting compound selected to inhibit agents interfering with ability of the growth factor to regulate or mediate the above processes.

Representative examples of imaging agents suitable to be incorporated in envelope 14 include, without limitation, Roentgen contrast agent, MRI contrast agent, and ultrasound contrast agent.

The pharmaceutical agent(s) may be incorporated into the fibers of envelope 14 in more than one way. For example, when the fibers are formed via a spinning technique, the agent(s) can be mixed with the liquefied polymer used in the spinning process.

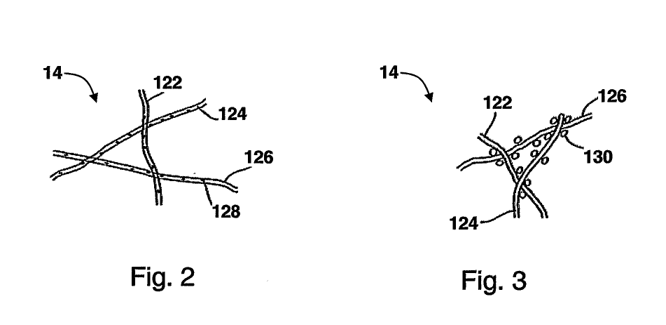

FIG. 2 illustrates another technique for incorporating the pharmaceutical agents into envelope 14. Shown in FIG. 2 is a portion of a non-woven web of polymer fibers produced according to a preferred embodiment of the present invention. Fibers 122, 124 and 126 intersect and are joined together at the intersections, the resultant interstices rendering the web highly porous. The fibers are preferably, thin so as provide envelope 14 with a large surface area which allows a high quantity of pharmaceutical agents to be incorporated thereon. When the fibers are electrospun fibers, their surface area approaches that of activated carbon, thereby making the non-woven web of polymer fibers an efficient local drug delivery system. In the representative illustration shown in FIG. 2, the agents(s) are constituted by particles 128 embedded in the polymer fibers. This embodiment is particularly useful when the pharmaceutical agent is a medicament which is to be released during the first post-operative days and weeks. The duration of the delivery process is effected by the type of polymer used for fabricating the envelope. Specifically, optimal release rate is ensured by using moderately stable biodegradable polymers.

FIG. 3 illustrates an alternative technique for incorporating the agent(s) into envelope 14. In this embodiment, the agent(s) are constituted by compact objects 130, distributed between the polymer fibers of the envelope. Compact objects 130 may be in any known form, such as, but not limited to, moderately stable biodegradable polymer capsules.

When it is desired to incorporate a pharmaceutical agent which is released over a prolong period of time (e.g., several months to several years) the agent(s) are dissolved or encapsulated in a layer made of biostable fibers. The rate of diffusion from within a biostable layer is substantially slower, thereby ensuring a prolonged effect of release.

Thus, the time scale of substance release is controlled according to various exemplary embodiments of the present invention by the type of polymer, the technique in which the pharmaceutical agent(s) are introduced into the polymer fibers, and the concentration of the pharmaceutical agent.

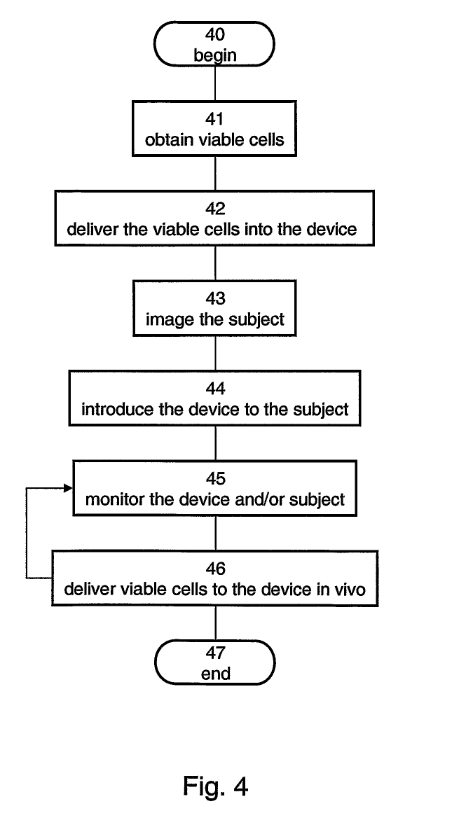

Reference is now made to FIG. 4 which is a flowchart diagram of a method suitable for cell grafting, according to various exemplary embodiments of the present invention. The method can be used for delivering a biologically active substance to a subject (a human or other mammalian subject). The method can be also used for providing a metabolic capability or function to a target tissue in the subject's body.

It is to be understood that, unless otherwise defined, the method steps described hereinbelow can be executed either contemporaneously or sequentially in many combinations or orders of execution. Specifically, the ordering of the flowchart diagram is not to be considered as limiting. For example, two or more method steps, appearing in the following description or in the flowchart diagram in a particular order, can be executed in a different order (e.g., a reverse order) or substantially contemporaneously. Additionally, several method steps described below are optional and may not be executed.

The method begins at step 40 and, optionally and preferably continues to optional step 41 in which a sufficient amount of viable cells (e.g., cells 22) is obtained. The cells can be harvested from a donor or they can be obtained from a cell bank. A cell bank is particularly useful when the cells are replicating cells or cell lines adapted for in vitro growth. The advantage of using a cell bank is that it is a source of cells prepared from the same culture or batch of cells. That is, all cells originated from the same source of cells and have been exposed to the same conditions and stresses. When the cells are harvested from a donor, the donor can be the subject itself, a different subject of the same species, or a different subject of a different species, depending on the type of cells which are used.

The method, optionally and preferably, continues to optional step 42 in which the viable cells are delivered into a device having a compartment enclosed by a semipermeable envelope formed with at least one inlet port (e.g., device 10 as further detailed hereinabove). The viable cells can be delivered into the compartment of the device via the inlet port using an injection device (see, e.g., FIG. 1b), such as a syringe or the like.

The method continues to optional step 43 in which at least a part of the subject is imaged, e.g., by MRI, CT or ultrasound so as to identify the location in the subject's body which requires the biologically active substance. Thus, for example, when the location is in the brain, the head of the subject can be imaged by CT or MRI to determine the target site and stereotactic coordinates. In the embodiments in which the imaging step is employed, the envelope of the device preferably comprises an imaging agent as further detailed hereinabove.

The method continues to step 44 in which the device is introduced to the location which requires the biologically active substance, preferably in proximity to the target tissue. The device can be introduced to the selected location using any known procedure, including, non-invasive, minimally invasive and invasive procedures. In various exemplary embodiments of the invention percutaneous injection, and more preferably image guided percutaneous injection, is employed. When the selected location is external, e.g., a cutaneous wound, the device can be used as a wound dressing and the procedure can be fully non-invasive.

The method optionally and preferably continues to step 45 in which the device and/or the subject are monitored. The monitoring can be visual, e.g., by imaging or it can include more involved tests and analyses. For example, the monitoring can include biopsy of the tissue adjacent to the device and/or extraction of fluids from the device in vivo via the inlet port using a syringe or other similar device. Step 45 is preferably performed during a predetermined latency period, which can be from several hours to several days, weeks or months.

The method can continue to optional step 46 in which a sufficient amount of cells and/or a cell medium are delivered into the compartment of the device in vivo, i.e., while the device is at the implantation location. The cells and a cell medium can be delivered via the inlet port of the device as further detailed hereinabove. When the implantation location is not external, the delivery of cells and/or cell medium is preferably image guided. The cells can be of the same type as the cells that were delivered at optional step 42 (in the embodiment in which step 42 is executed), or they can be of other type, depending on the decision of the physician. The cells can be harvested from a donor (which can be the subject itself, a different subject of the same species, or a different subject of a different species) or they can be obtained from a cell bank, as further detailed hereinabove. According to a preferred embodiment of the present invention the method loops back to step 45 (monitoring) periodically to assess performance of the device. Depending on the results of the monitoring the physician can decide whether or not to add or replace the viable cells and cell medium in the compartment of the device.

The method ends at step 47.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-46. (canceled)

47. A device for cell grafting, comprising a compartment enclosed by a semipermeable envelope made at least in part of nonwoven polymer fibers, and at least one inlet port formed in said envelope, said at least one inlet port being accessible from the exterior of said envelope and configured for establishing fluid communication with said compartment.

48. The device for cell grafting according to claim 47, wherein said device is further characterized by said semipermeable envelope comprising a plurality of layers, said envelope preferably having a molecular weight cutoff from about 75 kDa to about 500 kDa; further wherein said device comprises at least one inlet port defined by characteristics selected from a group consisting of generally closed and self sealing following piercing, non-woven swellable polymer fibers; formed in said envelope, hydrophilic; and configured for maintaining fluid homeostasis between said compartment and the environment or any combination thereof.

49. The device for cell grafting according to claim 47 wherein said semipermeable envelope comprises at least one pharmaceutical agent incorporated therein, further wherein said pharmaceutical agent is selected from a group consisting of antibodies immobilized upon said semipermeable envelope, a diagnostic agent, a pro-angiogenic agent, vascular endothelial growth factor, an antibiotic agent, an immuno-suppressing agent and at least one regulatory compound.

50. The device according to claim 47 wherein the device further comprises a sufficient amount of viable cells, preferably at least about 10,000 cells.

51. The device for cell grafting according to claim 49 wherein said regulatory compound comprises a potentiating compound selected to increase and/or mediate the ability of said growth factor to regulate or mediate at least one of: cell proliferation, cell differentiation, tissue regeneration and cell attraction and further and/or at least one regulatory compound comprises an inhibiting compound selected to inhibit agents interfering with ability of said growth factor to regulate or mediate at least one of: cell proliferation, cell differentiation, tissue regeneration and cell attraction.

52. The device for cell grafting according to claim 50 wherein said cells are selected from the group consisting of insulin producing cells, adrenal chromaffin cells, antibody-secreting cells, fibroblasts, astrocytes and beta cell lines and Islets of Langerhans.

53. The device for cell grafting according to claim 50 wherein said cells are capable of secreting a biologically active substance capable of providing a biological function to a subject.

54. The device for cell grafting according to claim 53 wherein said biologically active substance is selected from a group consisting of analgesic or a pain-reducing substances, growth factor trophic factors, nerve growth factors, basic fibroblast growth factors, a platelet derived growth factors, epidermal growth factors neurotrophic factors ciliary neurotrophic factors, brain-derived neurotrophic factors glial-derived neurotrophic factos neurotrophin-enkephalins peptides, catecholamine peptides, opioid peptides a hormone, cytokine, lymphokine neuropeptide neurotensin and Substance P.neurotransmitter, dopamine, L-dopa, gamma aminobutyric acid, serotonin, acetylcholine, noradrenaline, epinephrine, glutamic acid, parathyroid hormone, interleukin, erythropoietin, albumin, transferrin and Factor VIII or any combination thereof.

55. A method of delivering a biologically active substance to a mammal, comprising delivering into the device of claim 47 a sufficient amount of viable cells being capable of secreting the biologically active substance, and introducing the device to a location requiring the biologically active substance.

56. The method of claim 55, wherein said method further comprises steps of
delivering a sufficient amount of viable cells, preferably about 10,000 prior to said introducing of said device to said location while device is at said location; said introducing optionally comprising percutaneously injecting the device to said mammal;
harvesting said viable cells from a mammalian donor; imaging at least a part of said body during said step of introducing the device to said location; and
monitoring the device in vivo.

57. The method of claim 56 wherein said semipermeable envelope comprises at least one pharmaceutical agent incorporated therein, further wherein said pharmaceutical agent is selected from a group consisting of antibodies immobilized upon said semipermeable envelope, a diagnostic agent, a pro-angiogenic agent, vascular endothelial growth factor, an antibiotic agent, an immuno-suppressing agent and at least one regulatory compound.

58. The method of claim 57 wherein said method comprises steps of providing said at least one regulatory compound selected to increase and/or mediate the ability of said growth factor to regulate or mediate at least one of: cell proliferation, cell differentiation, tissue regeneration and cell attraction and optionally providing at least one regulatory compound selected to inhibit agents interfering with ability of said growth factor to regulate or mediate at least one of: cell proliferation, cell differentiation, tissue regeneration and cell attraction.

59. The method of claim 55 wherein said cells are selected from the group consisting of insulin producing cells, adrenal chromaffin cells, antibody-secreting cells, fibroblasts, astrocytes and beta cell lines and Islets of Langerhans.

60. The method of claim 55 wherein said cells are capable of secreting a biologically active substance capable of providing a biological function to a subject.

61. The method of claim 55 wherein said biologically active substance is selected from a group consisting of analgesic or a pain-reducing substances, growth factor trophic factors, nerve growth factors, basic fibroblast growth factors, a platelet derived growth factors, epidermal growth factors neurotrophic factors ciliary neurotrophic factors, brain-derived neurotrophic factors glial-derived neurotrophic factos neurotrophin-enkephalins peptides, catecholamine peptides, opioid peptides a hormone, cytokine, lymphokine neuropeptide neurotensin and Substance P.neurotransmitter, dopamine, L-dopa, gamma aminobutyric acid, serotonin, acetylcholine, noradrenaline, epinephrine, glutamic acid, parathyroid hormone, interleukin, erythropoietin, albumin, transferrin and Factor VIII or any combination thereof.
